SEQUENCE LISTING (62 sequences)

SEQ ID NO: 1 - DNA, 24 bp, Artificial
  Prime sequence
attccttcccagatggagactatg

SEQ ID NO: 2 - DNA, 24 bp, Artificial
  Prime sequence
gccctcattagtccttggctctta

SEQ ID NO: 3 - DNA, 24 bp, Artificial
  Prime sequence
ttcatgcttttcagacctctcttc

SEQ ID NO: 4 - DNA, 24 bp, Artificial
  Prime sequence
aatatgagctaccacccaagagga

SEQ ID NO: 5 - DNA, 24 bp, Artificial
  Prime sequence
caccattcgatttggccaattcct

SEQ ID NO: 6 - DNA, 24 bp, Artificial
  Prime sequence
acactctgtggctattttctttta

SEQ ID NO: 7 - DNA, 24 bp, Artificial
  Prime sequence
aagttctaccccagaacatgacta

SEQ ID NO: 8 - DNA, 24 bp, Artificial
  Prime sequence
cccacttagcctggcttaaccaag

SEQ ID NO: 9 - DNA, 24 bp, Artificial
  Prime sequence
gagggccgggttgggtgtcttacc

SEQ ID NO: 10 - DNA, 24 bp, Artificial
  Prime sequence
acccggtggggaacaaccagacgg

SEQ ID NO: 11 - DNA, 24 bp, Artificial
  Prime sequence
ctctgccctctctccctctggccc

SEQ ID NO: 12 - skipped

SEQ ID NO: 13 - DNA, 24 bp, Artificial
  Prime sequence
ctcagtctttcagccccatttgag

SEQ ID NO: 14 - DNA, 24 bp, Artificial
  Prime sequence
ccgaccgctcgggagctcgccccc

SEQ ID NO: 15 - DNA, 24 bp, Artificial
  Prime sequence
cagcaattgtgagcatatcagcat

SEQ ID NO: 16 - DNA, 24 bp, Artificial
  Primer sequence
ggacttccttaaactttaaccgaa

SEQ ID NO: 17 - DNA, 24 bp, Artificial
  Primer sequence
tgaaattttgctttataggagaaa

SEQ ID NO: 18 - DNA, 51 bp, Homo sapiens
gattccttcccagatggagactatggtcccaacctggaagcagctgccccc

SEQ ID NO: 19 - DNA, 51 bp, Homo sapiens
ctaaggaagggtctacctctgataccacggttggaccttcgtcgacggggg

SEQ ID NO: 20 - DNA, 20 bp, Artificial
  Primer sequence
tccccctcaactgagaactc

SEQ ID NO: 21 - DNA, 20 bp, Artificial
  Primer sequence
aaggctgagccataccagac

SEQ ID NO: 22 - DNA, 20 bp, Artificial
  Primer sequence
caaggccagtgacccccata

SEQ ID NO: 23 - DNA, 20 bp, Artificial
  Primer sequence
catggcaccgtttggttcag

SEQ ID NO: 24 - DNA, 20 bp, Artificial
  Primer sequence
atcgacttcgacttcgcacc

SEQ ID NO: 25 - DNA, 20 bp, Artificial
  Primer sequence
acgttcatactgctgtggag

SEQ ID NO: 26 - DNA, 20 bp, Artificial
  Primer sequence
acaggggccaccattcgatt

SEQ ID NO: 27 - DNA, 20 bp, Artificial
  Primer sequence
tgtgctcaggttcccagttg

SEQ ID NO: 28 - DNA, 20 bp, Artificial
  Primer sequence
gggaagtcagtgcagctcaa

SEQ ID NO: 29 - DNA, 18 bp, Artificial
  Primer sequence
cccagcgtgtttttcctg

SEQ ID NO: 30 - DNA, 20 bp, Artificial
  Primer sequence
aagctcttctggagggcagt

SEQ ID NO: 31 - DNA, 20 bp, Artificial
  Primer sequence
gacaccttcacgttgtcctg

SEQ ID NO: 32 - DNA, 20 bp, Artificial
  Primer sequence
agttccacgtggccctacta

SEQ ID NO: 33 - DNA, 20 bp, Artificial
  Primer sequence
cggcatcagatcttcatcct

SEQ ID NO: 34 - DNA, 20 bp, Artificial
  Primer sequence
ttcctctacatgggggtcac

SEQ ID NO: 35 - DNA, 20 bp, Artificial
  Primer sequence
cacagtgaggatgaggacga

SEQ ID NO: 36 - DNA, 24 bp, Artificial
  Primer sequence
cttccttgagatcttggcttccta

SEQ ID NO: 37 - DNA, 20 bp, Artificial
  Primer sequence
attgcaatgcaggccagaga

SEQ ID NO: 38 - DNA, 20 bp, Artificial
  Primer sequence
ggagctgcagagagaaagga

SEQ ID NO: 39 - DNA, 19 bp, Artificial
  Primer sequence
ctcagagccctgcatctca

SEQ ID NO: 40 - DNA, 22 bp, Artificial
  Primer sequence
gcaatatgctttatggtccgct

SEQ ID NO: 41 - DNA, 21 bp, Artificial
  Primer sequence
tcagaggcaagaattcctcca

SEQ ID NO: 42 - DNA, 21 bp, Artificial
  Primer sequence
gcttcctagaggaatcgaagg

SEQ ID NO: 43 - DNA, 26 bp, Artificial
  Primer sequence
gtttcctatatcacacaggtgtcctc

SEQ ID NO: 44 - DNA, 28 bp, Artificial
  Primer sequence
ctaatggtaagactgacacattacctca

SEQ ID NO: 45 - DNA, 21 bp, Artificial
  Primer sequence
cctggtacagtgaaacgatgg

SEQ ID NO: 46 - DNA, 30 bp, Artificial
  Primer sequence
aaaaaaattccttcccagatggagactatg

SEQ ID NO: 47 - DNA, 35 bp, Artificial
  Primer sequence
aaaaaaaaaaagccctcattagtccttggctctta

SEQ ID NO: 48 - DNA, 40 bp, Artificial
  Primer sequence
aaaaaaaaaaaaaaaattcatgcttttcagacctctcttc

SEQ ID NO: 49 - DNA, 45 bp, Artificial
  Primer sequence
aaaaaaaaaaaaaaaaaaaaaaatatgagctaccacccaagagga

SEQ ID NO: 50 - DNA, 50 bp, Artificial
  Primer sequence
aaaaaaaaaaaaaaaaaaaaaaaaaacaccattcgatttggccaattcct

SEQ ID NO: 51 - DNA, 55 bp, Artificial
  Primer sequence
aaaaaaaaaaaaaaaaaaaaaaaaaaaaaaaacacgctgtggctattttgtttta

SEQ ID NO: 52 - DNA, 60 bp, Artificial
  Primer sequence
aaaaaaaaaaaaaaaaaaaaaaaaaaaaaaaaaaaaaagttctaccccagaacatgacta

SEQ ID NO: 53 - DNA, 65 bp, Artificial
  Primer sequence
aaaaaaaaaaaaaaaaaaaaaaaaaaaaaaaaaaaaaaaaacccacttagcctggcttaaccaag

SEQ ID NO: 54 - DNA, 30 bp, Artificial
  Primer sequence
aaaaaagagggccgggttgggtgtcttacc

SEQ ID NO: 55 - DNA, 35 bp, Artificial
  Primer sequence
aaaaaaaaaaaacccggtggggaacaaccagacgg

SEQ ID NO: 56 - DNA, 40 bp, Artificial
  Primer sequence
aaaaaaaaaaaaaaaactctgccctctctccctctggccc

SEQ ID NO: 57 - DNA, 45 bp, Artificial
  Primer sequence
aaaaaaaaaaaaaaaaaaaaatgctgtgggtggtgaagtccacgc

SEQ ID NO: 58 - DNA, 50 bp, Artificial
  Primer sequence
aaaaaaaaaaaaaaaaaaaaaaaaaactcagtctttcagccccatttgag

SEQ ID NO: 59 - DNA, 60 bp, Artificial
  Primer sequence
aaaaaaaaaaaaaaaaaaaaaaaaaaaaaaaaaaaaccgaccgctcgggagctcgccccc

SEQ ID NO: 60 - DNA, 65 bp, Artificial
  Primer sequence
aaaaaaaaaaaaaaaaaaaaaaaaaaaaaaaaaaaaaaaaacagcaattgtgagcatatcagcat

SEQ ID NO: 61 - DNA, 30 bp, Artificial
  Primer sequence
aaaaaaggacttccttaaactttaaccgaa

SEQ ID NO: 62 - DNA, 35 bp, Artificial
  Primer sequence
aaaaaaaaaaatgaaattttgctttataggagaaa

(19) United States
(12) Patent Application Publication
(10) Pub. No.: US 2009/0104612 A1
(43) Pub. Date: Apr. 23, 2009

(54) DETECTION OF BLOOD GROUP GENES

(75) Inventors: Marion Reid, Jamaica, NY (US); Asok Chaudhuri, Hicksville, NY (US)

Correspondence Address: K&L Gates LLP, 1900 MAIN STREET, SUITE 600, IRVINE, CA, 92614-7319, US

(73) Assignee: New York Blood Center, Inc., New York, NY (US)

(21) Appl. No.: 12/254,707

(22) Filed: Oct. 20, 2008

Related U.S. Application Data
(60) Provisional application No. 60/981,283, filed on Oct. 19, 2007.

(51) Int. Cl. C12Q 1/68; C07H 21/04

(52) U.S. Cl. 435/6; 536/23.1

(57) ABSTRACT

Disclosed herein are nucleic acid molecules which permit the accurate and direct determination of blood groups based on the presence of certain genes. A method of determining blood groups is also provided.

### CROSS REFERENCE TO RELATED APPLICATIONS

This application claims the benefit under 35 U.S.C. §119(e) to U.S. Provisional Patent Application No. 60/981,283 filed Oct. 19, 2007, the entire contents of which is incorporated by reference herein.

### STATEMENT REGARDING FEDERALLY SPONSORED RESEARCH

This invention described in this application was made with funds from the National Institutes of Health, Grant Number P50 HL54459, and the sponsor being SCOR (HLB-NIH). The United States Government has certain rights in this invention.

### FIELD OF THE INVENTION

The present inventions relates to methods for detecting blood group genes with specific isolated nucleic acids.

### BACKGROUND OF THE INVENTION

The determination of blood group types in both patients and donors is of paramount importance in transfusion medicine. Transfusion of mismatched red blood cells (RBCs) to patients can cause severe hemolytic and inflammatory reactions, which could be fatal. Blood transfusions can be made safe by minimizing the risk of adverse transfusion reactions, which are triggered when antibodies circulating in the patient's blood stream encounter antigens displayed on a donor's red blood cells.

Besides ABO and Rh blood group antigens, knowledge of other blood group antigens is important for blood transfusions in patients who are chronically transfused, especially those with hemophilia and sickle cell anemia. Moreover, complete blood group antigen profiling is important to search for antigen-negative red blood cells in antigen negative patients for transfusion or in patients with null phenotypes.

Although serologic typing and labeling methods exist for some blood group antigens, typing for other blood group antigens is limited. These limitations include escalating costs of commercial reagents, lack of sufficient amount of appropriate antibodies, and labor intensive red blood cell testing and data entry. As a result, most donor centers screen only a selected cohort of donor blood and maintain a limited inventory of more fully phenotyped or genotyped units. This practice has the potential to introduce delays in treatment, which can create significant additional expense in patient care and can exacerbate emergency situations.

Typing for blood group antigens is usually determined by the classical (antigen-antibody) agglutination reactions using specific alloantibodies. Although the agglutination reactions can be dependable, their execution and interpretation require skilled technical experience. Accordingly, the resultant typing for blood group antigens can be subjective depending on the expertise of the technician. Moreover, the alloantibodies used are occasionally not specific enough, which leads to misinterpretation of the results. In addition, the availability and supply sources of some of these reagents and antibodies are limited.

Currently, various methods of DNA based technology have been developed to determine blood group genes present in patients/donors. Many DNA-based assays, such as SS-PCR, polymerase chain reaction-restriction fragment length polymorphism (PCR-RFLP) and microchips have been described to study variant blood group genes. One disadvantage of these assays is that they depend on indirect evidence for the presence of genes.

### SUMMARY OF THE INVENTION

In one embodiment, an isolated nucleic acid molecule is provided. The nucleic acid molecule includes a sequence having 15 to 33 nucleotides. The sequence also has a sequence identity of at least 70% to a sequence selected from the group consisting of SEQ ID NO: 1 through 17.

In an embodiment, the nucleic acid molecule is DNA, such as a probe primer. In one embodiment, the nucleic acid molecule is modified. The modification can include, for example, a 5′-end “tail” having varying lengths of a non-human sequence. Methods of designing and creating the modified 5′-end tail are well known in the art. Examples of such modifications include a poly-A tail, or a poly-GATC tail of varying lengths.

In another embodiment, nucleic acid molecule further includes a detectable label. Such detection molecules are known and include, for example, spectroscopic, photochemical, biochemical, immunochemical, or chemical means. For example, useful labels include ³²P, fluorescent dyes, electron-dense reagents, reporter molecules such as enzymes (as commonly used in ELISAs), biotin, haptens or proteins for which antisera or monoclonal antibodies are available. Such labels are detectable by known means.

In another embodiment, a method is provided for determining at least one blood group. The method includes obtaining blood from a patient or donor; extracting a nucleic acid fragment from the blood, the detection of which permits differentiation of alleles of common blood groups; amplifying the nucleic acid fragment; and detecting the nucleic acid fragment by hybridizing the fragment with a probe primer having 15 to 33 nucleotides and a sequence identity of at least 70% to a sequence selected from the group consisting of SEQ ID NO: 1 through 17.

Common blood groups are known in the art. In one embodiment, the blood groups include, but are not limited to, Duffy, Duffy-GATA, FYX, Dombrock, Landsteiner-Wiener, Colton, Scianna, Diego, Kidd, Lutheran, MNS, Kell, MNS, and Ss.

The nucleic acid fragment can be, for example, genomic DNA or cDNA. In one embodiment, the nucleic acid contains a single nucleotide polymorphism (SNP). The presence of a particular SNP is correlated with a known blood group.

In another embodiment, a plurality of nucleic acid fragments are detected concurrently.

### BRIEF DESCRIPTION OF THE FIGURES

FIG. 1 depicts the principle of the SNaPshot™ method exemplified with Duffy blood group genes, FYA and FYB. Asterisks represents the added nucleotides during extension reactions. Representative fluorescent peaks are shown below each incorporated nucleotide.

FIG. 2 depicts GeneMapper™ electropherogram results for sample P876 (FIG. 2A-2C) and P8 (FIG. 2D-2F) at all 17 SNPs in 10 blood groups. Probe primers are labeled above each bin in gray boxes, and alleles are identified below each peak within the bin. FIGS. 2A and 2D. Multiplex I. FIGS. 2B and 2E. Multiplex II. FIGS. 2C and 2F. Multiplex Ill.

FIG. 3 depicts a schematic diagram for the disclosed method for blood group gene detection.

### DETAILED DESCRIPTION OF THE INVENTION

### Isolated Nucleic Acid Molecules

In one aspect of the present disclosure, an isolated nucleic acid molecule is provided. The nucleic acid molecule of the invention is a single-stranded oligomer of deoxyribonucleic acid (DNA) that includes a minimum number of 15 nucleotides, a minimum of 17, or a minimum of 21 nucleotides. The isolated nucleic acid molecule comprises a maximum number of 33 nucleotides, a maximum of 31, or a maximum of 26 nucleotides. A suitable range of minimum and maximum numbers of nucleotides may be obtained by combining any of the above minima with any of the above maxima. In one embodiment, the nucleic acid molecules contain 24 nucleotides.

The isolated nucleic acid molecule may be obtained from or derived by known methods from natural sources. Alternatively, the isolated nucleic acid molecule may be produced synthetically according to methods known in the art. Such methods include, for example, cloning and restriction of appropriate sequences and direct chemical synthesis by a method such as the phosphotriester method of Narang et al. (Meth Enzvmol 68:90 (1979); the phosphodiester method of Brown et al. (Meth Enzymol 68:109 (1979); the diethylphosphoramidite method of Beaucage et al. (Tetrahedron Lett 22:1859 (1981); and the solid support method in U.S. Pat. No. 4,458,066. It is preferred that the isolated nucleic acid molecule be at least substantially purified to avoid introduction of artifacts into the genotype determination method.

The isolated nucleic acid molecule may differ from a comparative sequence selected from the group consisting of SEQ ID NOs: 1 through 17 by having one or more substitutions, additions, deletions, and/or mismatches. The nucleic acid molecules also include a sequence identity of at least 70% to a sequence selected from the group consisting of SEQ ID NOs: 1 through 17.

TABLE 1
Exemplary Primers

| Primer | Primer | | Length |
|---|---|---|---|
| name | sequence 5′→3′ | SEQ ID NO: | (bp) |
| PFYA/B | ATT CCT TCC CAG ATG | SEQ ID NO:1 | 24 |
| | GAG ACT ATG | | |
| PGATA | GCC CTC ATT AGT CCT | SEQ ID NO:2 | 24 |
| | TGG CTC TTA | | |
| PFYX | TTC ATG CTT TTC AGA | SEQ ID NO:3 | 24 |
| | CCT CTC TTC | | |
| PDOA/B1 | AAT ATG AGC TAC CAC | SEQ ID NO:4 | 24 |
| | CCA AGA GGA | | |
| PDOA/B2 | CAC CAT TCG ATT TGG | SEQ ID NO:5 | 24 |
| | CCA ATT CCT | | |
| PDOA/B3 | ACA CTC TGT GGC TAT | SEQ ID NO:6 | 24 |
| | TTT CTT TTA | | |
| PDOJOA | AAG TTC TAC CCC AGA | SEQ ID NO:7 | 24 |
| | ACA TGA CTA | | |
| PDOHY | CCC ACT TAG CCT GGC | SEQ ID NO:8 | 24 |
| | TTA ACC AAG | | |
| PLWAB | GAG GGC CGG GTT GGG | SEQ ID NO:9 | 24 |
| | TGT CTT ACC | | |
| PCOAB | ACC CGG TGG GGA ACA | SEQ ID NO:10 | 24 |
| | ACC AGA CGG | | |
| PSC | CTC TGC CCT CTC TCC | SEQ ID NO:11 | 24 |
| | CTC TGG CCC | | |
| PDI | TGC TGT GGG TGG TGA | SEQ ID NO:12 | 24 |
| | AGT CCA CGC | | |
| PJKA/B | CTC AGT CTT TCA GCC | SEQ ID NO:13 | 24 |
| | CCA TTT GAG | | |
| PLUMB | CCG ACC GCT CGG GAG | SEQ ID NO:14 | 24 |
| | CTC GCC CCC | | |
| POYPAM/N | CAG CAA TTG TGA GCA | SEQ ID NO:15 | 24 |
| | TAT CAG CAT | | |
| PK | GGA CTT CCT TAA ACT | SEQ ID NO:16 | 24 |
| | TTA ACC GAA | | |
| PGYPBS/s | TGA AAT TTT GCT TTA | SEQ ID NO:17 | 24 |
| | TAG GAG AAA | | |

Similarly, nucleic acid probes can be made directed to be the opposite strand of the target DNA. Such probes to the complementary strand of the target DNA will read from the 3′ end to 5′end of the sense strand. As for an example, the double strand genomic DNA sequence for FY is as follows:

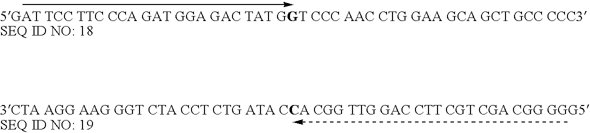

A nucleic acid probe primer sequence (SEQ ID NO: 1) for Duffy blood group A is shown above by solid arrow over a portion of the top sequence (SEQ ID NO:18) and reads a portion of the Duffy blood group A antigen as 5′GAT 7CC TTC CCA GAT GGA GAC TAT G. A probe primer from the complementary DNA strand can be generated as shown by a broken arrow at the bottom of a portion of the lower sequence (SEQ ID NO:19) and reads a portion of the Duffy blood group A antigen as 5′GGG GGC AGC TGC TTC CAG GTT GGC A3′. In this case, the polymorphic nucleotide (the nucleotide overlap between the two primers) will be read as either “G” or “C” for FYA depending on the primer used. Utilizing this strategy, for FYB, the polymorphic nucleotide will be read as either “A” or “T”. Similar approach and probe construction design can be utilized for each blood group genes by locating the polymorphic nucleotide and based on the complementary genomic or cDNA sequences.

The primer probes of SEQ ID NOs: 1 through 17 can be utilized to locate these primer probes to the complementary strand.

In one embodiment, the isolated nucleic acid molecule comprises a nucleotide sequence that is at least 70%, at least 80%, at least 90%, at least 95%, or at least 99% identical to the total number of nucleotides in a sequence selected from a group consisting of SEQ ID NOs: 1 through 17.

Percent sequence identity is calculated as the percent nucleotides that are identical in two sequences being compared. The determination of identity (percent) may be carried out as follows: I=(n÷24)×100, wherein I is the identity in percent and n is the number of identical nucleotides between the isolated nucleic acid molecule and the comparative sequence selected from the group consisting of SEQ ID NOs: 1 through 17.

For example, if the isolated nucleic acid molecule had 24 nucleotides and had 12 identical nucleotides compared to a comparative sequence selected from the group consisting of SEQ ID NOs: 1 through 17, the percent identity would be 50% (i.e., {12/24}×100). Alternatively, if the isolated nucleic acid molecule had 24 nucleotides and had 20 identical nucleotides compared to a comparative sequence selected from the group consisting of SEQ ID NOs: 1 through 17, the percent identity would be 83% (i.e., {20/24}×100).

In one embodiment, in addition to the percent identity described above, the isolated nucleic acid molecule has a sequence identity of 100% to the 3′ end of the respective sequence selected from a group consisting of SEQ ID NOs: 1 through 17. Specifically, the isolated nucleic acid molecule has a sequence identity of 100% to at least three nucleotides starting from the 3′ end of the respective sequence and reading sequentially towards the 5′ end of the respective sequence. In another embodiment, the isolated nucleic acid molecule has a sequence identity of 100% to at least six nucleotides, and to at least nine nucleotides, starting from the 3′ end of the respective sequence and reading sequentially towards the 5′ end of the respective sequence.

In one embodiment, the isolated nucleic acid molecule is a probe primer. As further described below, such probe primers can be effective in identifying single nucleotide polymorphisms (SNPs) that are associated with particular blood group genes.

The primer hybridizes to a desired template, such as DNA, genomic DNA, cDNA, RNA, or fragments thereof.

The term hybridization refers to the degree of base-pairing between two nucleic acids as described above. As is known to one of skill in the art, adenine (A) can form hydrogen bonds or base pair with thymine (T) and guanine (G) can form hydrogen bonds or base pair with cytosine (C). Thus, A is complementary to T and G is complementary to C.

The probe primer sequence is not required to represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The probe primer, however, must be of sufficient complementarity to the desired template in order to prime the synthesis of a desired extension product. For example, there may be any number of base pair mismatches that interfere with base pairing between the target sequence and the primer. If the number of mutations is so great that no base pairing can occur under even the least stringent of base pairing conditions, the sequence is not a complementary target sequence.

It should be noted in this context that “mismatch” is a relative term and meant to indicate a difference in the identity of a base at a particular position, termed the “detection position” herein, between two sequences. In general, sequences that differ from wild type sequences are referred to as mismatches. However, and particularly in the case of SNPs, what constitutes “wild type” may be difficult to determine as multiple alleles can be relatively frequently observed in the population, and thus “mismatch” in this context requires the artificial adoption of one sequence as a standard. Thus, for the purposes of this disclosure, sequences are referred to herein as “perfect match” and mismatch”. “Mismatches” are also sometimes referred to as “allelic variants”.

Accordingly, the probe primer must be of sufficient complementarity to be able to anneal with the desired template strand in a manner sufficient to provide the 3′ hydroxyl moiety of the primer in appropriate juxtaposition for use in the initiation of synthesis by a polymerase or similar enzyme.

As described above, the isolated nucleic acid molecule preferably has a sequence identity of 100% to the 3′ end of the respective sequence selected from a group consisting of SEQ ID NOs: 1 through 17. Consequently, the isolated nucleic acid molecules have a sufficient complementarity to anneal with the desired template strand in a manner sufficient to provide the 3′ hydroxyl moiety of the primer in appropriate juxtaposition for use in the initiation of synthesis by a polymerase or similar enzyme.

The criteria for designing sequence-specific primers are well known to persons of skill in the art. The sequence-specific portions of the primers are of sufficient length to permit specific annealing to complementary sequences in ligation products and amplification products, as appropriate.

In a further embodiment, the isolated nucleic acid molecule is a modified probe primer. A modified probe primer, as used herein, is a probe primer with a 5′-end “tail” having varying lengths of a non-human sequence. Methods of designing and creating the modified 5′-end tail are well known in the art. Examples of such modifications include a poly-A tail, poly-G tail, poly-C tail, poly-T tail, or a poly-GATC tail, or a combination thereof. The modifications can add varying lengths of nucleotides to the primer.

The tail lengths can vary by increments of a number of nucleotides that can be optimized by one of ordinary skill in the art. For example, the tail lengths can vary by increments of 5 or 4 nucleotides. In one embodiment, the tail comprises 3 to 54 nucleotides. In another embodiment, the tail comprises 6 to 48 nucleotides, 12 to 42 nucleotides, 18 to 36 nucleotides, or 24 to 32 nucleotides in length. In additional embodiments the tail comprises 6, 11, 16, 21, 26, 31, 36, 41, 46 or 51 nucleotides.

Such modified probe primers allow for target sequences to be more easily differentiated, especially in situations where multiple targets are being hybridized. Examples of modified probe primers include SEQ ID NOs: 46 through 62.

In yet a further embodiment, the isolated nucleic acid molecule is a detectable probe primer. The term “detectable probe primer” refers to a probe primer that includes a detectable label. The label can be detected by, for example, spectroscopic, photochemical, biochemical, immunochemical, or chemical means. For example, useful labels include, but are not limited to, radioactive labels, fluorescent labels, electron dense labels, enzymes, biotin, haptens and proteins. Non-limiting examples include ³²P, fluorescent dyes, electron-dense reagents, reporter molecules such as enzymes (as commonly used in ELISAs), biotin, or haptens or proteins for which antisera or monoclonal antibodies are available. Such labels are detectable by known means.

Method for Determining Blood Group

In one embodiment disclosed herein, a method is provided for determining blood group genes, comprising obtaining blood from a patient or donor, extracting a nucleic acid fragment from the blood, the detection of which permits differentiation of alleles of common blood groups, amplifying the nucleic acid fragment, and detecting the nucleic acid fragment by hybridizing said fragment with a probe primer having 15 to 33 nucleotides and a sequence identity of at least 70% to a sequence selected from the group consisting of SEQ ID NO: 1 through 17.

Blood group antigens are inherited, polymorphic, structural characteristics located on proteins, glycoproteins, or glycolipids on the exofacial surface of the red blood cell membrane. Common blood groups are known in the art. Common blood groups include, for example, the following groups: Duffy, Dombrock, Landsteiner-Wiener, Colton, Scianna, Diego, Kidd, Lutheran, MNS, Kell, MNS, and Ss.

The majority of polymorphic blood group antigens arise from single nucleotide polymorphisms (SNPs) of blood group genes. SNPs identified within the variant blood group genes have encoded missense mutations, nonsense mutations, alternative splicing and silencing of the gene promoter. Examples of SNPs in the respective blood group systems are listed in Table 2.

TABLE 2
Single nucleotide polymorphisms in blood group systems
and the corresponding amino acid changes and antigens.

| | GenBank | | | | |
|---|---|---|---|---|---|
| Blood group | Accession | Location | SNP site | Amino acid | Antigens |
| Duffy | AL035403 | Exon 2 | Fy125G > A | Gly42Asp* | Fy^{a}/Fy^{b} |
| | | Promoter | Fy-33T > C | — | Silencing Fy |
| | | region | | | |
| | | Exon 2 | 265C > T | Arg89Cys | Fy^{x}[Fy(b+^{w})) |
| Dombrock | AY899803 | Exon 2 | DO793A > G | Asn265Asp | Do^{a}/Do^{b} |
| | | | DO6247 > C | Leu208Leu | Do^{a}/Do^{b} |
| | | | DO378C > T | Tyr126Tyr | Do^{a}/Do^{b} |
| | | | DO350C > T | Thr11711e | Jo(a+)/Jo(a−) |
| | | | DO323G > T | Gly108Val | Hy+/Hy |
| Landsteiner- | X93093 | Exon 1 | LW308A > G | Gln70Arg | LW^{a}/LW^{b} |
| Wiener | | | | | |
| Colton | M77829 | Exon 1 | CO134C > T | Ala45Va1 | Co^{a}/Co^{b} |
| Scianna | AJ505036 | Exon 3 | SC169G > A | Gly57Arg | Sc1/Sc2 |
| Diego | X77738 | Exon 19 | D12561T > C | Leu854Pro | Di^{a}/Di^{b} |
| Kidd | NT_010966 | Exon 9 | 838G > A | Asp280Asn | Jk^{a}/Jk^{b} |
| Lutheran | NT_011109 | Exon 3 | 230A > G | His77Arg | Lu^{a}/Lu^{b} |
| MNS | L31856 | Exon 2 | 59C > T | SerlLeu | GYPA (M/N) |
| Kell | AY228336 | Exon 6 | 578MC | Met193Thr | K/k |
| MNS | M60708 | Exon 4 | 143T > C | Met29Thr | GYPB (S/s) |

*The amino acid changes are expressed using the three letter code for the wild type amino acid followed by the position number of the amino acid and followed by the three letter code for changed amino acid.

The appropriate DNA sequences encompassing the SNPs above are available in GenBank database under the accession numbers listed above.

The claimed methods include obtaining blood from a patient or donor. The blood sample can be obtained by any method known to those in the art. Suitable methods include, for example, venous puncture of a vein to obtain a blood sample. The blood can be obtained from any tissue of the patient or donor.

The patient or donor can be any human. For example, the patient can be a patient in need of a blood transfusion, a chronically transfused patient, patients with blood related diseases such as sickle cell disease, or any person in need of a blood group determination.

The methods also include extracting a nucleic acid fragment from the blood, the detection of which permits differentiation of alleles of common blood groups.

The nucleic acid fragment can be derived from DNA or RNA. The DNA or RNA can be isolated from the blood sample by any method known to those in the art. For example, commercial kits can be used to isolate DNA, such as the QIAGEN System (QIAmp DNA Blood Mini Kit, Valencia, Calif.) or automated DNA extraction BioRobot M96 (Qiagen) with beads (MagAttract, Qiagen), or RNA isolation using TRIZOL® total RNA isolation reagent.

The claimed methods can optionally further comprise a rapid estimation of DNA concentration by ethidium bromide dot quantitation methods, which are well known in the art. For example, the concentrations of the extracted DNA from the blood samples can be estimated by a comparison to the intensity of ethidium bromide-stained DNA standards of known concentrations under ultraviolet (UV) light.

The extracted nucleic acid fragment can be DNA, for example, genomic DNA, or cDNA derived from mRNA. The mRNA can be isolated from reticulocytes or peripheral blood. Tumor call lines that express blood group antigens may also be the source of the extracted nucleic acid fragment, e.g., the source of RNA. An example of such a tumor cell line is human K562, which is deposited in the ATCC.

Detection of the extracted nucleic acid fragment permits differentiation of alleles of common blood groups. The detection includes hybridizing and amplifying the extracted nucleic acid fragment with sense and antisense primers having 15 to 33 nucleotides and a sequence identity of at least 70% to a sequence selected from the group consisting of SEQ ID NO: 1 through 17.

With regard to detection of the extracted nucleic acid fragment, the DNA is optionally amplified by methods known in the art. One suitable method is the polymerase chain reaction (PCR) method described in U.S. Pat. No. 4,683,195, which is incorporated by reference herein for all it contains regarding the polymerase chain reaction. For example, oligonucleotide primers complementary to a nucleotide sequence flanking and/or present at the site of the SNP of the allele can be used to amplify the allele.

The extracted nucleic acid fragment is used to determine whether an allele containing a SNP is present in the blood sample. The presence of an allele containing a SNP can be determined by any method known to those skilled in the art, such as use of oligonucleotides and polymerase chain reaction (PCR).

Methods of optimizing amplification reactions are well known to those skilled in the art. For example, it is well known that PCR can be optimized by altering times and temperatures for annealing, polymerization, and denaturing, as well as changing the buffers, salt, and other regions in the reaction composition. Optimization can also be affected by the design of the amplification primers used. For example, the length of the primers, as well as the G-C to A-T ratio can alter the efficiency of primer annealing, thus altering the amplification reaction.

In one embodiment, oligonucleotide PCR primers that can be used for amplification of blood group genes include oligonucleotides comprising a sequence identity of at least 70% to a sequence selected from the group consisting of SEQ ID NOs: 20 through 45 (Table 3). The oligonucleotide PCR primers disclosed herein can also be used in multiplex PCR reactions. These PCR primers are also a novel aspect of the invention and uniquely enhance the amplification of blood group genes.

TABLE 3
Oligonucleotide PCR primers for amplification of blood group genes.

| | PCR | | | | | Product |
|---|---|---|---|---|---|---|
| Blood | Primer | Sequence | | Length | Tm | size |
| group | name | 5′→3′ | SEQ ID NO: | (bases) | (° C.) | (bp) |

Multiplex I

| | | | | | | |
|---|---|---|---|---|---|---|
| Duffy | Fy-1 | TCC CCC TCA ACT GAG AAC TC | SEQ ID NO.20 | 20 | 59 | 392 |
| | Fy-2 | AAG GCT GAG CCA TAC CAG AC | SEQ ID NO:21 | 20 | 59 | |
| | GATA-1 | CAA GGC CAG TGA CCC CCA TA | SEQ ID NO:22 | 20 | 60 | 189 |
| | GATA-2 | CAT GGC ACC GTT TGG TTC AG | SEQ ID NO:23 | 20 | 65 | |
| Dombrock | Do-1N | ATC GAC TTC GAC TTC GCA CC | SEQ ID NO:24 | 20 | 63 | 300 |
| | Do-2N | ACG TTC ATA CTG CTG TGG AG | SEQ ID NO:25 | 20 | 56 | |
| | Do-3 | ACA GGG GCC ACC ATT CGA TT | SEQ ID NO:26 | 20 | 66 | 240 |
| | Do-4 | TGT GCT CAG GTT CCC AGT TG | SEQ ID NO:27 | 20 | 62 | |

Multiplex II

| | | | | | | |
|---|---|---|---|---|---|---|
| Landsteiner- | LW-1 | GGG AAG TCA GTG CAG CTC AA | SEQ ID NO:28 | 20 | 62 | 187 |
| Weiner | LW-2 | CCC AGC GTG TTT TTC CTG | SEQ ID NO:29 | 18 | 60 | |
| Colton | Co-1 | AAG CTC TTC TGG AGG GCA GT | SEQ ID NO:30 | 20 | 61 | 137 |
| | Co-2 | GAC ACC TTC ACG TTG TCC TG | SEQ ID NO:31 | 20 | 59 | |
| Scianna | Sc-1 | AGT TCC ACG TGG CCC TAC TA | SEQ ID NO:32 | 20 | 60 | 170 |
| | Sc-2 | CGG CAT CAG ATC TTC ATC CT | SEQ ID NO:33 | 20 | 60 | |
| Diego | DI-1N | TTC CTC TAC ATG GGG GTC AC | SEQ ID NO:34 | 20 | 60 | 392 |
| | RDIN | CAC AGT GAG GAT GAG GAC GA | SEQ ID NO:35 | 20 | 60 | |
| Kidd | Kidd-1N | CTT CCT TGA GAT CTT GGC TTC | SEQ ID NO:36 | 24 | 62 | 216 |
| | | CTA | | | | |
| | Kidd-2 | ATT GCA ATG CAG GCC AGA GA | SEQ ID NO:37 | 20 | 64 | |
| Lutheran | FLU | GGA GCT GCA GAG AGA AAG GA | SEQ ID NO:38 | 20 | 60 | 143 |
| | RLUN | CTC AGA GCC CTG CAT CTC A | SEQ ID NO:39 | 19 | 60 | |
| MN | GYPA-1N | GCA ATA TGC TTT ATG GTC CGC T | SEQ ID NO:40 | 22 | 62 | 341 |
| | GYPA-2 | TCA GAG GCA AGA ATT CCT CCA | SEQ ID NO:41 | 21 | 62 | |

Multiplex III

| | | | | | | |
|---|---|---|---|---|---|---|
| Kell | Kell-1N | GCT TCC TAG AGG AAT CGA AGG | SEQ ID NO:42 | 21 | 59 | 392 |
| | Kell-2N | GTT TCC TAT ATC ACA CAG GTG | SEQ ID NO:43 | 26 | 61 | |
| | | TCC TC | | | | |
| Ss | GYPB-1N | CTA ATG GTA AGA CTG ACA CAT | SEQ ID NO:44 | 28 | 61 | 220 |
| | | TAC CTC A | | | | |
| | GYPB-2N | CCT GGT ACA GTG AAA CGA TGG | SEQ ID NO:45 | 21 | 60 | |

The methods may be conducted in single or multiplex reactions. As used herein, the term “multiplex” refers to multiple reactions occurring in the same reaction container. Different combinations of multiplex PCRs can be performed to obtain a robust amplification for each amplicon. As used herein, “amplicon” refers to pieces of DNA formed as the products of natural or artificial amplification events. For example, they can be formed via polymerase chain reactions (PCR) or ligase chain reactions (LCR), as well as by natural gene duplication. Oligonucleotides set forth in SEQ ID NOs: 20 through 45 can be used in the multiplex PCR reactions.

The methods also include detecting the nucleic acid fragment by hybridizing the fragment with a modified probe primer as described above. Examples of a modified probe primer include SEQ ID NOs: 46 through 62 (Table 4). SEQ ID NOs: 46 through 62 are identical to SEQ ID NOs: 1 through 17 with the addition of a poly-A tail. The modified probe primer can then be used for detection of SNPs in blood groups. The oligonucleotides set forth in SEQ ID NOs: 46 through 62 were designed to be used as probes for detecting each respective SNP and to stop at a position 5′ of the respective SNP site.

The sequences of the primers that can be used for detection of SNPs in blood groups, the lengths of their poly-A tails for multiplexing, and optimized annealing temperatures that can be used in the methods of the claimed invention are provided below in Table 4.

The primers used in hybridizing preferably can further include a detectable label, an optical label such as a fluorophore, into the amplicon for detection. The incorporation of a label into these primers allows for direct detection of blood group genes in a sample based on based on direct visualization of nucleotide present at the respective SNP sites.

The nucleic acid molecules described above, more specifically, the primers and probe primers, can be utilized to detect genes associated with common blood groups using any known gene-based detection system. Some gene-based detection systems utilize the presence of SNP, to detect blood group genes.

TABLE 4

The oligonucleotide modified probe primers used for detection of
SNPs in blood groups.

| | | | | | Final |
|---|---|---|---|---|---|
| Modified | | | | | concentration |
| probe | | | | | in |
| primer | Modified probe primer sequence | | Length | Tm | SNaPshot ™ |
| name | 5′→3′ | SEQ ID NO: | (bp) | (° C.) | reaction |
| pFYA/B | 6 As + ATT CCT TCC CAG ATG GAG ACT ATG | SEQ ID NO:46 | 30 (6 As + 24) | 67 | 20 nM |
| pFY-GATA | 11 As + GCC CTC ATT AGT CCT TGG CTC TTA | SEQ ID NO:47 | 35 (11 As + 24) | 71 | 20 nM |
| pFYX | 16 As + TTC ATG CTT TTC AGA CCT CTC TTC | SEQ ID NO:48 | 40 (16 As + 24) | 71 | 40 nM |
| pDOA/BI | 21 As + AAT ATG AGC TAC CAC CCA AGA GGA | SEQ ID NO:49 | 45 (21 As + 24) | 73 | 10 nM |
| pDOA/B2 | 26 As + CAC CAT TCG ATT TGG CCA ATT CCT | SEQ ID NO:50 | 50 (26 As + 24) | 77 | 50 nM |
| Pdoa/B3 | 31 As + ACA CGC TGT GGC TAT TTT GTT TTA | SEQ ID NO:51 | 55 (31 As + 24) | 75 | 50 nM |
| pDOJOA | 36 As + AAG TTC TAC CCC AGA ACA TGA CTA | SEQ ID NO:52 | 60 (36 As + 24) | 75 | 50 nM |
| pDOHY | 41 As + CCC ACT TAG CCT GGC TTA ACC AAG | SEQ ID NO:53 | 65 (41 As + 24) | 78 | 50 nM |
| pLWA/B | 6 As + GAG GGC CGG GTT GGG TGT CTT ACC | SEQ ID NO:54 | 30 (6 As + 24) | 75 | 20 nM |
| pCOA/B | 11 As + ACC CGG TGG GGA ACA ACC AGA CGG | SEQ ID NO:55 | 35 (11 As + 24) | 79 | 200 nM |
| pSC | 16 As + CTC TGC CCT CTC TCC CTC TGG CCC | SEQ ID NO:56 | 40 (16 As + 24) | 78 | 10 nM |
| pDI | 21 As + TGC TGT GGG TGG TGA AGT CCA CGC | SEQ ID NO:57 | 45 (21 As + 24) | 79 | 150 nM |
| pJKA/B | 26 As + CTC AGT CTT TCA GCC CCA TTT GAG | SEQ ID NO:58 | 50 (26 As + 24) | 76 | 30 nM |
| pLUA/B | 36 As + CCG ACC GCT CGG GAG CTC GCC CCC | SEQ ID NO:59 | 60 (36 As + 24) | 84 | 30 nM |
| pGYPAM/N | 41 As + CAG CAA TTG TGA GCA TAT CAG CAT | SEQ ID NO:60 | 65 (41 As + 24) | 77 | 200 nM |
| pK/k | 6 As + GGA CTT CCT TAA ACT TTA ACC GAA | SEQ ID NO:61 | 30 (6 As + 24) | 64 | 200 nM |
| pGYPBS/s | 11 As + TGA AAT TTT GCT TTA TAG GAG AAA | SEQ ID NO:62 | 35 (11 As + 24) | 65 | 100 nM |

Methods for analyzing SNPs are well known in the art and can be conducted in a single or multiplex reaction. Examples of such methods include as allele specific hybridization, primer extension reactions, minisequencing, MALDI-TOF mass spectrometry (MS), pyrosequencing, microarrays and fluorescence detection, allele specific oligonucleotide ligation, invasive cleavage; electrophoresis and fluorescence detection. See for example, Hashmi, et al. “A flexible array format for large-scale, rapid blood group DNA typing. Transfusion. 2005 May; 45(5):680-8, and Sobrino, et al, “SNPs in forensic genetics: a review on SNP typing methodologies,” Forensic Sci Int. 2005 Nov. 25; 154(2-3):181-94.

Preferably, a multiplex SNP analysis is performed. Most preferably, a multiplex SNP analysis is performed with a commercial kit, such as the SNaPshot™ multiplex kit (Applied Biosystems). The SNaPshot™ kit is based on a minisequencing reaction followed by electrophoresis and fluorescence detection. In minisequencing, a primer anneals to its target DNA immediately adjacent to the SNP and is then extended by a DNA polymerase with a single nucleotide that is complementary to the polymorphic site.

Multiple assay reactions can be performed simultaneously, i.e., multiplexed, by appropriately designing the 5′ end tails of primers used in a SNP analysis. Examples of primers with appropriately designed 5′ end tails for SNP analysis include the modified probe primers described above, for example, SEQ ID NOs: 46 through 62. Such primers are useful where quantification is to be carried out by capillary or gel electrophoresis.

The tails of differing length cause the corresponding product sequences to form bands at different locations in the gel or capillary. The presence of these bands at different locations permit the corresponding nucleotide differences in the DNA being analyzed to be identified. Such primers are also useful to avoid overlap between the final SNaPshot™ products.

In one embodiment, the primer comprises varying lengths of poly-A tails at the 5′ end in order to create probe primers of varying sizes to be used in multiplex reaction settings. For example, the primer can vary by increments of five nucleotides through the addition of poly-A tails to the 5′ end.

In another embodiment, the primer comprises varying lengths of poly-GATC tails at the 5′ end in order to create probe primers of varying sizes to be used in multiplex reaction settings. For example, the primer can vary by increments of four nucleotides through the addition of poly-GATC tails to the 5′ end.

The products are then separated electrophoretically in an automated capillary DNA sequencer or other suitable sequencer.

Many steps are required to perform multiplex reactions, such as the design and concentration of the primers for PCR and minisequencing reactions of each set of SNPs and optimizing temperature cycles. The design and concentration of the primers for PCR and minisequencing reactions of each set of SNPs have been described in detail above. With respect to optimized temperature cycles, the annealing temperature for the complementary region between any primer described herein and its corresponding template should be at least 40° C., at least 45° C., or at least 50° C., and should be at most 55° C., at most 65° C., or at most 72° C.

A high degree of multiplexing will dramatically reduce the cost of screening nucleic acid samples containing a large number of SNPs with alleles to be analyzed.

### EXAMPLE 1

### DNA Preparation

Twenty nine human blood samples were obtained from volunteer blood donors at the New York Blood Center (NYBC) with informed consent approved by the institutional review board.

Genomic DNA from 200 μl of blood sample was extracted using the QIAamp DNA Blood Mini Kit protocol (QIAGEN Inc.). Twenty microliters of proteinase K was added to 200 μl of sample and 200 μl of Buffer AL and mixed by pulse-vortexing for 15 sec and incubated at 56° C. for 10 min. Tubes were briefly centrifuged and 200 μl of absolute ethanol was added to each sample, mixed by pulse-vortexing for 15 sec, and briefly centrifuged. This mixture was transferred to the QIAamp Spin Column in a 2 ml collection tube, and centrifuged at 8,000 rpm for 1 min. The column was placed in a new 2 ml collection tube, 500 μl of Buffer AW1 was added, and the column centrifuged at 8,000 rpm for 1 min. After the column was placed in a new collection tube, 500 μl of Buffer AW2 was added, and the column was centrifuged at 14,000 rpm for 3 min. The column was placed in a new collection tube and centrifuged at 14,000 rpm for 1 min and placed in a new 1.5 ml microcentrifuge tube for elution of DNA with 200 μl of Buffer AE by incubating at room temperature for 5 min and centrifuging at 8,000 rpm for 1 min. DNA samples were then kept at −20° C. for long-term storage.

### EXAMPLE 2

### Rapid Estimation of DNA Concentration by Ethidium Bromide Dot Quantitation

The concentrations of the extracted DNA in all blood samples were estimated by comparison to the intensity of ethidium bromide-stained DNA standards of the following concentrations: 0 μg/ml, 1 μg/ml, 2.5 μg/ml, 5 μg/ml, 7.5 μg/ml, 10 μg/ml and 20 μg/ml.

Four microlilters of a 1 pg/ml ethidium bromide solution was added to 4 μl of each standard and to 4 μl of each sample and all mixtures were spotted side by side on plastic wrap and placed on a UV transilhuninator (AlphaImager™ 2200, Alpha Innotech Co., San Leandro, Calif.). The intensity of the ethidium bromide-stained DNA standards and samples were visualized under UV light and a photograph was taken for record keeping. The intensity of signal from each sample was quantified using a computer program known in the art and the values for the intensity of the DNA standards were used to plot a standard curve of DNA concentration. This standard curve was used to determine the specific concentration of DNA in each extraction from blood samples to allow equal amounts of DNA template to be amplified in each subsequent PCR reaction.

### EXAMPLE 3

### PCR Primer and Probe Primer Design for Snapshot Assay

Based on the knowledge of molecular bases of blood group antigens, the multiplex SNaPshot™ method was used to detect single nucleotide polymorphisms (SNP) at known locations of 10 blood group systems, namely, MNS, Lutheran, Kell, Duffy, Kidd, Diego, Scianna, Dombrock, Colton and Lansteiner-Wiener. The appropriate DNA sequences encompassing the 17 SNPs analyzed are available in GenBank database and the accession numbers are shown in Table 2.

The 17 SNPs associated with antigens and amino acid changes in 10 blood group systems art also shown in Table 2. The SNaPshot method can detect more than one SNPs in a single tube based on the dideoxy single-base extension of an unlabeled oligonucleotide primer. Initially different combinations of multiplex PCRs were done to obtain robust amplifications for each amplicon. The forward and reverse primers for each gene are shown in Table 3.

The primers to be used as probes for the detection of each individual SNP were designed to stop just 5′ of each SNP site. The length of each probe primer includes 24 nucleotides specific to the sequence of the gene, plus varying lengths of poly-A tails at the 5′ end to create probes of varying sizes to be used in a multiplex reaction setting. Each probe primer included in a single multiplex reaction in this study varied by increments of S nucleotides through the addition of poly-A tails to the 5′ end in order to avoid overlap between the final SNaPshot products. The annealing temperature for the complementary region between any primer and its corresponding template should be at least 50° C. Sequences of each probe primer, the lengths of their poly-A tails and annealing temperatures are given in Table 4. Three multiplex PCR amplification reactions were optimized as described below.

### EXAMPLE 4

### Template Preparation, Multiplex PCR and Purification of PCR Products

Genomic DNA regions surrounding each SNP site were amplified by three reactions of multiplex PCR. Multiplex I included primers to amplify regions containing SNPs in the Duffy and Dombrock blood groups; multiplex II included primers to amplify regions containing SNPs in the Lansteiner-Weiner, Colton, Scianna, Diego, Kidd, Lutheran and MN blood groups; and multiplex III included primers to amplify regions containing SNPs in the Kell and Ss blood groups. Oligonucleotide primer sequences for multiplex I, multiplex II and multiplex III PCR reactions are given in Table 3.

For each multiplexed PCR reaction, 100 ng of DNA was amplified by 5 U of Taq DNA polymerase (HotStarTaq, QIAGEN Inc.) in a 50 μl reaction mixture containing 2.5 mM MgCl₂, 1×PCR buffer, 0.2 mM dNTPs and 100 ng of pooled forward and reverse primers. PCR amplification was performed in a thermal cycler (9700, Perkin Elmer, Norwalk, Conn.) by the following conditions: 35 cycles of 94° C. for 20 seconds, 55° C. for 20 seconds and 72° C. for 30 seconds; followed by a final extension of 10 min at 72° C. PCR products of multiplex I and multiplex III were analyzed on a 1.2% agarose gel, while PCR products of multiplex II were analyzed on an 8% polyacrylamide gel. The PCR product sizes of multiplex II were closer to each other than other multiplexes. Therefore, it was suitable to used 8% polyacrylamide gel to separate PCR products in multiplex II because it provided higher resolution than 1.2% agarose gel. After PCR amplification, the PCR products were purified to remove excess dNTPs and primers using a modified protocol for ExoSAP-IT (USB Corporation, Cleveland, Ohio). Two microliters of ExoSAP-IT was added to 5 μl of PCR product and incubated at 37° C. for 1 hr, followed by enzyme inactivation at 75° C. for 15 min and storage at 4° C.

### EXAMPLE 5

### SNaPshot Multiplex Reaction Preparation

For all samples, 10 ng of purified PCR product and 10-200 ng of pooled probe primers for each multiplex SNP reaction were combined and brought to a volume of 5 μl with ddH₂O for the SNaPshot™ Multiplex SNP analysis reactions. In some cases, PCR products and probe primers were combined and totaled a volume greater than 5 μl and were then dried completely in a SpeedVac and resuspended in 5 μl ddH₂O. A positive control containing 2 μl of the supplied SNaPshot™ Multiplex Control Template and 1 μl of the SNaPshot™ Multiplex Control Primer Mix, and a negative control containing no template and 1 μl of the SNaPshot™ Multiplex Control Primer Mix were each brought up to a volume of 5 μl with ddH₂O to be run with each set of reactions. To each sample mixture, 5 μl of SNaPshot™ Multiplex Ready Reaction Mix was added on ice and samples were placed in a Bio-Red iCycler for 25 cycles of 10 sec at 96° C., 5 sec at 50° C. and 30 sec at 60° C. followed by rapid thermal ramp to 4° C. Post-extension treatment was conducted by adding 1 unit of shrimp alkaline phosphatase (USB Corporation) to each reaction and incubation at 37° C. for 1 hr followed by 15 min of enzyme deactivation at 75° C.

Following post-extension treatment, fresh tubes were prepared on ice containing 9 μl of Hi-Di Formamide (Applied Biosystems, Foster City, Calif.) and 0.5 μl of the GeneScan-120 Liz size standard. To these mixtures, 0.5 μl of each SNaPshot™ Multiplex PCR product was added and DNA was set to denature at 95° C. for 5 min and kept on ice before loading onto the ABI Prism 3100 Genetic Analyzer.

Samples were run using POP-4 polymer, 50 cm capillaries, an injection time of 30 sec and oven temperature of 60° C. Results were analyzed using GeneMapper™ Software v.3.5 (Applied Biosystems) in the SNaPshot™ default analysis method.

### EXAMPLE 6

### Identification of Blood Groups SNPs by Probe Primer Hybridization and Extension

After multiplex PCR of the 17 SNPs were performed, the 17 probe primers were hybridized to the 5′ end of the SNPs in the presence of fluorescently-labeled ddNTPs each having a different fluorescent color. The AmpliTaq DNA polymerase extended the primer by one nucleotide and adding only a single ddNTP to its 3′ end corresponding to the polymorphic nucleotide. The mobility of an oligonucleotide in capillary electrophoresis was determined by its size, nucleotide composition, and dye.

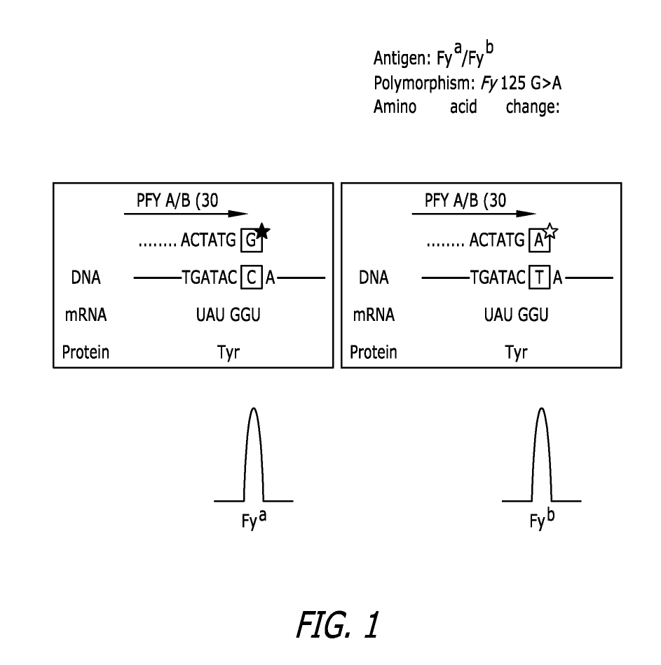

Each SNP probe primer is of known size, and was extended by one dideoxynucleotide base at the known SNP site. Therefore it is possible to distinguish each SNP from the rest in the multiplex by matching the migration (in base pairs) of each probe primer and the fluorescent dye specific to each base (A, C, G, or T) to the known probe primer size and the known allelic polymorphism. For example, the pFYA/B probe primer to detect the polymorphism in the Duffy gene at position 125 in exon 2 was 30-bp long and should detect homozygous G or A, or heterozygous G/A. Therefore, blue (G) or/and green (A) peaks which migrate at around 30-bp on the electropherogram are tracked and their identity as pFYA/B are determined using known controls. For each SNP the exact position of migration of each of the two possible alleles is determined and this bin position is applied to every reaction. The GeneMapper software would “call” each base that fell within these bins at the SNP sites. In FIG. 1, the basic principle of the SNaPshot™ method is shown based on the Fy^{a}/Fy^{b} blood group polymorphism.

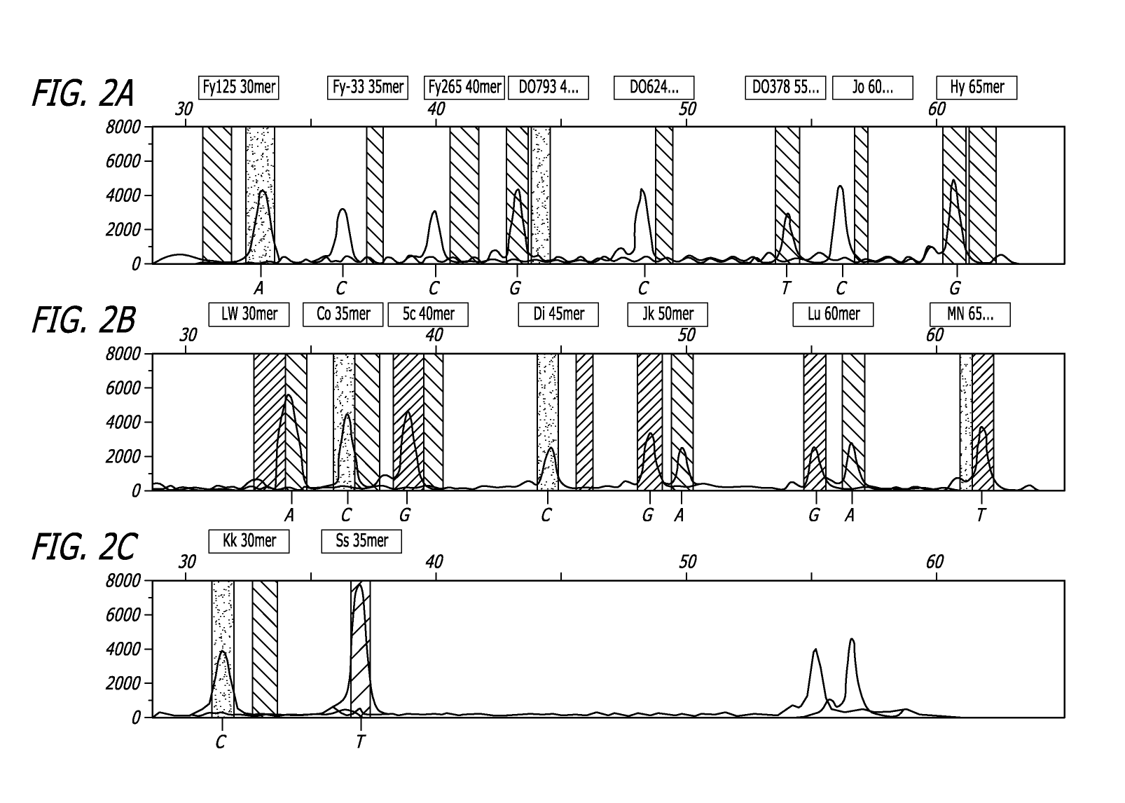

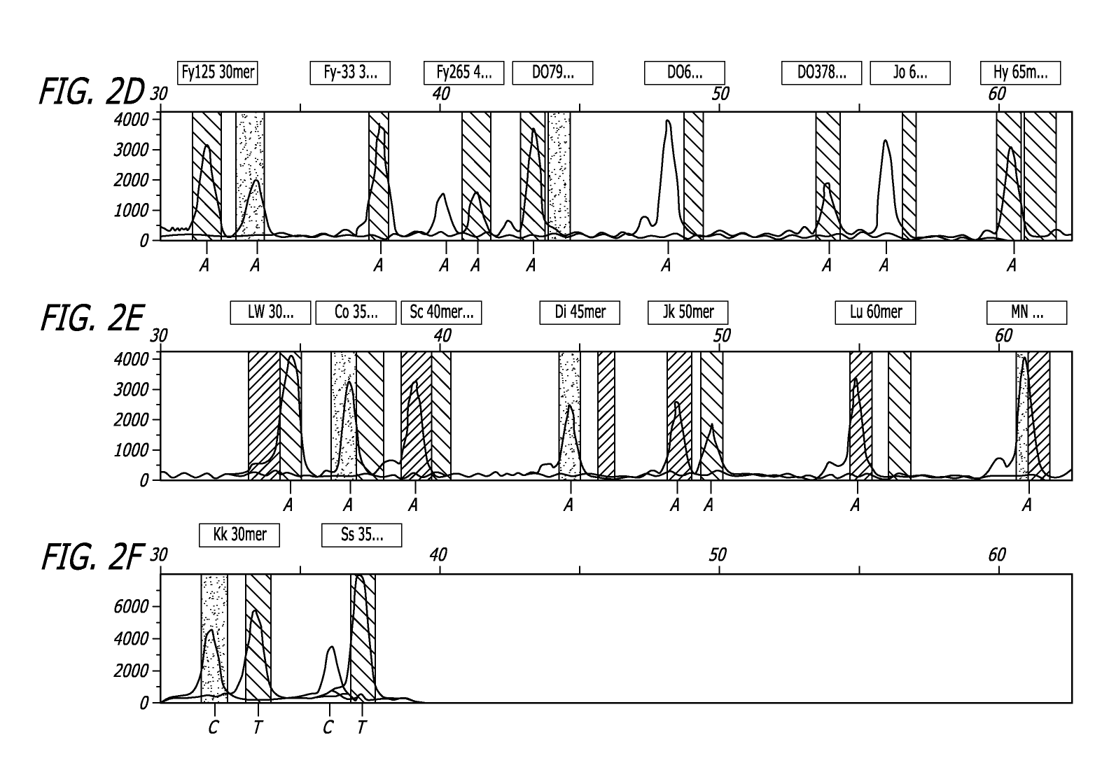

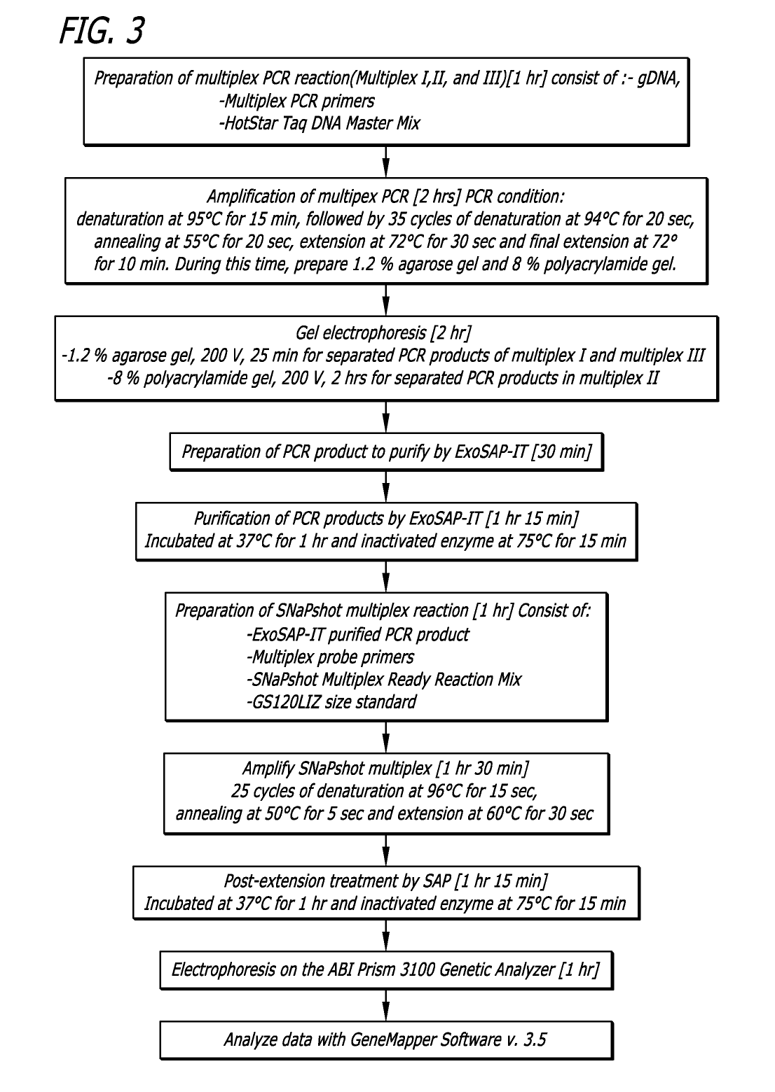

The concentrations of DNA template and SNaPshot™ probe primer within each of the three multiplex reactions were optimized (Table 3) to obtain a peak height of at least 800 and not greater than 8000. Probe concentration varied from 10 nM to 200 nM (Table 3). In general, the longer the probe length, the higher the concentration of the probe required to get the optimum signal. However, this is not true in all cases, as in the case of K/k and S/s, the probe concentration needed was 200 nM and 100 nM, respectively even though the probe length was 30 and 35 bp. The probe concentration needed might be dependent on the probe length and its base composition. Peak signals greater than around 7000 register as “off-scale” and create “pull-up peak” noise underneath the peak of interest. This occurrence does not usually interfere with the base-calling as long as peaks are kept below 8000. Peak signals less than 800 are considered not strong enough for a true allele call and are counted as “noise” should they happen to fall inside of the bins created to detect each polymorphism. FIG. 2 shows an example of data created for a representative sample in multiplex I, II, and III. Highlighted regions are bins created for each SNP and the allele present in this particular sample is called (A, C, G, or T) below the peak.

Seventeen SNP sites in 29 blood samples previously phenotyped and/or genotyped by either serology or microarray analysis, respectively, were used as controls to test the accuracy and reproducibility of the three multiplex SNaPshot™ assays. Controls that were heterozygous at each SNP position for multiplex I, II and III were created in order to design “bins” to analyze unknown data. Bins are defined by the specific position in base pairs where each peak corresponding to a labeled dideoxynucleotide allele attached to a probe primer migrates on the elecropherogram of data created by the GeneMapper™ software from the data produced on the 3100 ABI Prism DNA sequencer.

The histograms of three multiplex reactions after capillary electrophoresis is shown in FIG. 2. Genes were called based on the extended nucleotides (SNPs) on the probe primer. Similar reactions were carried out for all 29 samples. The results were compared with the previously determined types. SNaPshot™ analyses predicted the 17 SNPs accurately for all 29 samples tested. Although the signal peak heights were different for different SNPs, they were clearly identifiable. Both homozygous and heterozygous SNPs were detected with equal confidence. Of the 493 (17×29) SNPs analyzed, 409 were homozygous and 84 were heterozygous SNPs (Table 5). Any combination or number of blood group SNPs can be used; however, they must be optimized for each combination.

Identification of blood group antibodies and the provision of antigen-negative blood form the bases for safe blood transfusion. Current practice involves typing by hemagglutination and labeling all donor blood for ABO and Rh(D) antigens, (Standards Committee of American Association of Blood Banks, 2003) while typing for other blood group antigens is limited due to a number of factors. These limitations include labor-intensive hemagglutination testing and data-entry as well as escalating cost of commercial reagents, and lack of sufficient volumes of appropriate antibodies. As a result, most donor centers screen only a selected cohort of donors and maintain a limited inventory of antigen-negative products. This practice can introduce delays in treatment, which can lead to significant additional expense in patient care. Multiplex SNaPshot™ technology has the potential to increase the inventory of antigen-negative blood. Thus, especially in chronically-transfused patients, the reduction or prevention of alloimmunization to ‘minor’ blood group antigens would be possible. Although the genotype may not reflect the phenotype, DNA analysis will identify the potential antigen-negative for confirmation by hemagglutination.

Critical to the reliability of the method presented here is the fact that unambiguous allele discrimination is maintained, and interference from other polymorphisms is minimized, even in a highly multiplexed reaction, by invoking a parallel format of enzyme-mediated detection probe elongation. Hence, bead-displayed probes containing variable 3′ termini matching either the normal or a variant allele, act as nested primers for the simultaneous elongation of matching probes by a DNA polymerase. Each bead produces an assay signal reflecting the incorporation of fluorescently-labeled dNTPs into the elongation products displayed on that bead. At the same time, the high redundancy of the representation of probes on multiple copies of the same bead type enhances the statistical reliability of the readings.

Using a microplate format, 96 DNA samples can be typed in approximately 4 hrs, with only about 1 hr of “hands-on” time. The rate of throughput is further increased by “staggering” assay protocol steps, and by parallel processing of multiple plates. Ninety six samples can be loaded onto a capillary gene sequencer. One single run requires around 4 hrs to complete. A simple algorithm can be developed based on the probe length and the added bases during extension reaction to “call” for the blood group SNP present in DNA from the blood donor. Because the results are interpreted by software, an interface to a donor database eliminates the manual entry of results.

The Bioarray GeneChip method is based on the nucleotide match of the detection probes at the SNP sites and by the incorporation of fluorescent nucleotide during elongation reaction. Also, it involves the coupling of each probe to a dye infused bead, followed by the manufacturing of silicone microchips (Blood Chip). The presence of genes is determined indirectly based on either positive or no elongation of the probe. The negative reaction can be attributed to many unknown factors. The results may not be unambiguous.

In contrast, the adapted SNaPshot method is simpler, direct and cheaper. This procedure requires only the synthesis of probe primers. All the other reagents are commercially available and quality controlled. It does not require the development of microbead coupled probes and Blood Chip, which are expensive. Moreover, the determination of genes is direct, that is, the nucleotide present at the SNP site is directly determined by the DNA sequencer and can be visualized. As there is no unreactive probe, all probes must incorporate the nucleotides at the SNP sites, corresponding to the gene present. This makes this method more reliable in gene prediction. In some blood group genes, the long length of probe primer may cause low peak height, such as Kell and Ss. Previously, the detection of Kell and Ss blood group genes has been included in Multiplex II. The probe primers to detect the polymorphism in the Kell and Ss genes were 55 bp and 70 bp long. The results revealed that both genes gave a low peak height. After the detection of these genes using Multiplex III and reducing length of probe primers to 30 bp for Kell and 35 bp for Ss, the resulted peak heights were increased.

TABLE 5
Number of heterozygotes and homozygotes detected
for 17 SNP sites in 29 blood samples
MULTIPLEX 1
Duffy (30 mer)

| | |
|---|---|
| G/A | 8 |
| A | 14 |
| G | 7 |

Duffy (35 mer)

| | |
|---|---|
| T/C | 2 |
| T | 20 |
| C | 7 |

Duffy (40 mer)

| | |
|---|---|
| C/T | 1 |
| C | 28 |
| T | 0 |

Dombrock (45 mer)

| | |
|---|---|
| A/G | 8 |
| A | 5 |
| G | 16 |

Dombrock (50 mer)

| | |
|---|---|
| T/C | 8 |
| T | 5 |
| C | 16 |

Dombrock (55 mer)

| | |
|---|---|
| C/T | 9 |
| T | 16 |
| C | 4 |

Dombrock (60 mer)

| | |
|---|---|
| C/T | 0 |
| T | 1 |
| C | 28 |

Dombrock (65 mer)

| | |
|---|---|
| G/T | 1 |
| T | 0 |
| G | 28 |

MULTIPLEX 2
Landsteiner-Wiener (30 mer)

| | |
|---|---|
| A/G | 1 |
| A | 27 |
| G | 1 |

Colton (35 mer)

| | |
|---|---|
| C/T | 3 |
| C | 25 |
| T | 1 |

Scianna (40 mer)

| | |
|---|---|
| G/A | 2 |
| G | 27 |
| A | 0 |

Diego (45 mer)

| | |
|---|---|
| T/C | 2 |
| C | 26 |
| T | 1 |

Kidd (50 mer)

| | |
|---|---|
| G/A | 9 |
| G | 13 |
| A | 7 |

Lutheran (40 mer/60 mer)

| | |
|---|---|
| A/G | 4 |
| A | 1 |
| G | 24 |

MNS (65 mer)

| | |
|---|---|
| C/T | 16 |
| C | 9 |
| T | 4 |

MULTIPLEX 3
Kell (30 mer/55 mer)

| | |
|---|---|
| T/C | 3 |
| C | 25 |
| T | 1 |

S/sMNS (35 mer/70 mer)

| | |
|---|---|
| T/C | 7 |
| T | 10 |
| C | 12 |
| TOTAL A/G heterozygotes = | 32 |
| TOTAL C/T heterozygotes = | 51 |
| TOTAL G/T heterozygotes = | 1 |
| Total # heterozygotes detected = | 84 |
| Total A homozygotes = | 54 |
| Total C homozygotes = | 180 |
| Total G homozygotes = | 116 |
| Total T homozygotes = | 59 |
| Total # homozygotes detected = | 409 |

By facilitating the design and validation of multiplexed assays to type additional antigens such as RH, HLA as well as viral genotypes, the method described here has the potential to permit the creation of diverse inventories of fully characterized blood units available for delivery.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An isolated nucleic acid molecule comprising a sequence of 15 to 33 nucleotides and at least 70 percent identity to a nucleic acid sequence selected from the group consisting of SEQ ID NO:1 through SEQ ID NO:17.

2. The isolated nucleic acid molecule of claim 1 wherein said nucleic acid molecule is DNA.

3. The isolated nucleic acid molecule of claim 1 wherein said nucleic acid molecule is a probe primer.

4. The isolated nucleic acid molecule of claim 1 wherein said nucleic acid further includes a modification at the 5′ end.

5. The isolated nucleic acid molecule of claim 4 wherein said modification comprises a poly-A tail or a poly-GATC tail from 3 nucleotides to 54 nucleotides in length.

6. The isolated nucleic acid molecule of claim 1 wherein said nucleic acid molecule further includes a detectable label.

7. The isolated nucleic acid molecule of claim 1 wherein said detectable label is selected from the group consisting of radioactive labels, fluorescent labels, electron dense labels, enzymes, biotin, haptens and proteins.

8. A method for determining at least one blood group type in a blood sample, said method comprising:
obtaining a blood sample from a patient or donor,
extracting at least one nucleic acid fragment from said blood, the detection of which permits differentiation of alleles of common blood groups,
amplifying said nucleic acid fragments,
detecting the nucleic acid fragments by hybridizing said fragments with at least one nucleic acid molecule comprising a sequence of 15 to 33 nucleotides and at least 70 percent identity to a nucleic acid sequence selected from the group consisting of SEQ ID NO:1 through SEQ ID NO:17; and
determining at least one blood group type of said blood sample.

9. The method of claim 8 wherein said primer probe further includes a modification at the 5′ end.

10. The method of claim 9 wherein said modification comprises a poly-A tail or a poly-GATC tail from 3 nucleotides to 54 nucleotides in length.

11. The method of claim 8 wherein said primer probe further includes a detectable label.

12. The method of claim 9 wherein said detectable label is selected from the group consisting of radioactive labels, fluorescent labels, electron dense labels, enzymes, biotin, haptens and proteins.

13. The method of claim 8 wherein said nucleic acid contains a single nucleotide polymorphism.

14. The method of claim 8 wherein a plurality of nucleic acid fragments are detected concurrently.
